(19) United States
(12) Patent Application Publication
(10) Pub. No.: US 2018/0113118 A1
(43) Pub. Date: Apr. 26, 2018

(54) ELECTRONIC NEURON PAIN ASSAY

(71) Applicant: StemoniX Inc., Eden Prairie, MN (US)

(72) Inventor: Robert John Petcavich, The Woodlands, TX (US)

(21) Appl. No.: 15/789,335

(22) Filed: Oct. 20, 2017

Related U.S. Application Data
(60) Provisional application No. 62/411,261, filed on Oct. 21, 2016.

(51) Int. Cl. G01N 33/50

(52) U.S. Cl. CPC G01N 33/5058; G01N 33/502

(57) ABSTRACT

A method for detecting electrical activity of electrically active cells exposed to one or more compounds is provided.

### CROSS-REFERENCE TO RELATED APPLICATIONS

This application claims the benefit of the filing date of U.S. application Ser. No. 62/411,261, filed on Oct. 21, 2016, the disclosure of which is incorporated by reference herein.

### BACKGROUND

Pain is a distressing feeling often caused by intense or damaging stimuli, such as stubbing a toe or burning a finger. The examples represent respectively the three classes of nociceptive pain—mechanical, thermal and chemical—and neuropathic pain. Because it is a complex, subjective phenomenon, defining pain has been a challenge. The international Association for the Study of Pain's widely used definition states: Pain is an unpleasant sensory and emotional experience associated with actual or potential tissue damage, or described in terms of such damage. In medical diagnosis, pain is a symptom.

Pain motivates the individual to withdraw from damaging situations, to protect a damaged body part while it heals, and to avoid similar experiences in the future. Most pain resolves once the noxious stimulus is removed and the body has healed, but it may persist despite removal of the stimulus and apparent healing of the body. Sometimes pain arises in the absence of any detectable stimulus, damage or disease. Simple pain medications are useful in 20% to 70% of cases.

Pain is the most common reason for physician consultation in most developed countries. It is a major symptom in many medical conditions, and can interfere with a person's quality of life and general functioning. Psychological factors such as social support, hypnotic suggestion, excitement, or distraction can significantly affect pain's intensity or unpleasantness. In some arguments put forth in physician—assisted suicide or euthanasia debates, pain has been used as an argument to permit terminally ill patients to end their lives.

Nociceptive pain is caused by stimulation of sensory nerve fibers that respond to stimuli approaching or exceeding harmful intensity nociceptors and may be classified according to the mode of noxious stimulation. The most common categories are “thermal” (e.g., heat or cold), “mechanical” (e.g., crushing, tearing, shearing, etc.) and “chemical” (e.g., iodine in a cut or chemicals released during inflammation). Some nociceptors respond to more than one of these modalities and are consequently designated polymodal.

Nociceptive pain may also be divided into “visceral”, “deep somatic” and “superficial somatic” pain. Visceral structures are highly sensitive to stretch, ischemia and inflammation but relatively insensitive to other stimuli that normally evoke pain in other structures, such as burning and cutting. Visceral pain is diffuse, difficult to locate and often referred to a distant, usually superficial, structure. It may be accompanied by nausea and vomiting and may be described as sickening, deep, squeezing, and dull. Deep somatic pain is initiated by stimulation of nociceptors in ligaments, tendons, bones, blood vessels, fasciae and muscles, and is dull, aching, poorly-localized pain. Examples include sprains and broken bones. Superficial pain is initiated by activation of nociceptors in the skin or other superficial tissue, and is sharp, well defined and clearly located. Examples of injuries that produce superficial somatic pain include minor wounds and minor (first degree) burns.

Neuropathic pain is caused by damage or disease affecting any part of the nervous system involved in bodily feelings (the somatosensory system). Peripheral neuropathic pain is often described as “burning”, “tingling”, “electrical”, “stabbing”, or “pins and needles”. Bumping the “funny bone” elicits acute peripheral neuropathic pain.

Phantom pain is pain felt in a part of the body that has been lost or from which the brain no longer receives signals. It is a type of neuropathic pain. Phantom limb pain is a common experience of amputees.

The prevalence of phantom pain in upper limb amputees is nearly 82%, and in lower limb amputees is 54% one study found that eight days after amputation, 72 percent of patients had phantom limb pain, and six months later, 65 percent reported it. Some amputees experience continuous pain that varies in intensity or quality; others experience several bouts a day, or it may occur only once every week or two. It is often described as shooting, crushing, burning or cramping. If the pain is continuous for a long period, parts of the intact body may become sensitized, so that touching them evokes pain in the phantom limb. Phantom limb pain may accompany urination or defecation.

Local anesthetic injections into the nerves or sensitive areas of the stump may relieve pain for days, weeks, or sometimes permanently, despite the drug wearing off in a matter of hours; and small injections of hypertonic saline into the soft tissue between vertebrae produces local pain that radiates into the phantom limb for ten minutes or so and may be followed by hours, weeks or even longer of partial or total relief from phantom pain. Vigorous vibration or electrical stimulation of the stump, or current from electrodes surgically implanted onto the spinal cord, all produce relief in some patients.

Paraplegia, the loss of sensation and voluntary motor control after serious spinal cord damage, may be accompanied by girdle pain at the level of the spinal cord damage, visceral pain evoked by a filling bladder or bowel, or, in five to ten per cent of paraplegics, phantom body pain in areas of complete sensory loss. This phantom body pain is initially described as burning or tingling but may evolve into severe crushing or pinching pain, or the sensation of fire running down the legs or of a knife twisting in the flesh. Onset may be immediate or may not occur until years after the disabling injury. Surgical treatment rarely provides lasting relief.

Psychogenic pain, also called psychalgia or somatoform pain, is pain caused, increased, or prolonged by mental, emotional, or behavioral factors. Headache, back pain, and stomach pain are sometimes diagnosed as psychogenic. Sufferers are often stigmatized, because both medical professionals and the general public tend to think that pain from a psychological source is not “real”. However, specialists consider that it is no less actual or hurtful than pain from any other source.

People with long-term pain frequently display psychological disturbance, with elevated scores on the Minnesota Multiphasic Personality Inventory scales of hysteria, depression and hypochondriasis (the “neurotic triad”). Some investigators have argued that it is this neuroticism that causes acute pain to turn chronic, but clinical evidence points the other way, to chronic pain causing neuroticism. When long-term pain is relieved by therapeutic intervention, scores on the neurotic triad and anxiety fall, often to normal levels. Self-esteem, often low in chronic pain patients, also shows improvement once pain has resolved.

The term ‘psychogenic’ assumes that medical diagnosis is so perfect that all organic causes of pain can be detected; regrettably, all too often, the diagnosis of neurosis as the cause of pain hides ignorance of many aspects of pain medicine.

Breakthrough pain is transitory acute pain that comes on suddenly and is not alleviated by the patient's regular pain management. It is common in cancer patients who often have background pain that is generally well-controlled by medications, but who also sometimes experience bouts of severe pain that from time to time “breaks through” the medication. The characteristics of breakthrough cancer pain vary from person to person and according to the cause. Management of breakthrough pain can entail intensive use of opioids, including fentanyl.

Inadequate treatment of pain is widespread throughout surgical wards, intensive care units, accident and emergency departments, in general practice, in the management of all forms of chronic pain including cancer pain, and in end of life care. This neglect is extended to all ages, from neonates to the frail elderly. African and Hispanic Americans are more likely than others to suffer needlessly in the hands of a physician; and women's pain is more likely to be undertreated than men's.

The abuse of and addiction to opioids such as heroin, morphine, and prescription pain relievers is a serious global problem that affects the health, social, and economic welfare of all societies. It is estimated that between 26.4 million and 36 million people abuse opioids worldwide, with an estimated 2.1 million people in the United States suffering from substance use disorders related to prescription opioid pain relievers in 2012 and an estimated 467,000 addicted to heroin. The consequences of this abuse have been devastating and are on the rise. For example, the number of unintentional overdose deaths from prescription pain relievers has soared in the United States, more than quadrupling since 1999. There is also growing evidence to suggest a relationship between increased non-medical use of opioid analgesics and heroin abuse in the United States.

To address the complex problem of prescription opioid and heroin abuse to treat pain in this country, the special character of this phenomenon should be recognized and considered, to both confront the negative and growing impact of opioid abuse on health and mortality, but also to preserve the fundamental role played by prescription opioid pain relievers in healing and reducing human suffering. That is, scientific insight must strike the right balance between providing maximum relief from suffering while minimizing associated risks and adverse effects.

### SUMMARY

The disclosure provides a method of fabricating an electronic assay that models human pain neurons, in one embodiment, dorsal root ganglion (DRG) neurons, that are one of the major pathways to transmitting pain signals to the brain. An array of multielectrodes, with DRGs disposed on the electrode surface, monitors activity such as DRG action potentials and/or signaling during baseline cell conditions, after stimulation with known or suspected pain inducing agents, and subsequent treatment with known or potential pain killing substances.

The disclosure also provides a method of fabricating an electronic assay that models human pain neurons, in one embodiment, a co-culture of neurons and astrocytes. An array of multielectrodes, with the cells in the co-culture disposed on the electrode surface, monitors activity such as action potentials and/or signaling during baseline cell conditions, after stimulation with known or suspected pain inducing agents, and subsequent treatment with known or potential pain killing substances. In one embodiment, a co-culture of neurons and astrocytes is in a ratio of 10:90, 20:80, 30:70, 40:60, 50:50, 60:40, 70:30, 80:20, or 90:10, e.g., a ratio of about 50:50. The use of such co-cultures allows for an adult, e.g., human, model for drug efficacy and toxicity testing and allows the cells to live for extended periods of time, e.g., >6 months whereas pure neuron cultures usually die within 2 weeks.

Thus, the disclosure provides a method of electronically monitoring and quantifying pain in an in vitro neuron model as an array assay to screen molecules for the modulation or elimination of pain. In one embodiment, a co-culture of human neurons and astrocytes is employed. In one embodiment, human DRG neurons are employed, e.g., DRG neurons prepared from induced pluripotent stem cells (iPScs). The cells bond to and are cultured on a multielectrode array plate. The action potentials and/or synchronicity of the cells is/are monitored, e.g., in real time, to create a baseline pain electronic signal, which is subsequently challenged with pain producing stimuli. Subsequently, the cells are exposed to molecules that reduce or eliminate pain-induced action potential neuron firing behavior as a screen for discovery of treatments for human pain.

In one embodiment, an electronic neuron pain assay is provided comprising a multielectrode array, dorsal root ganglion cells bonded to the electrode array, e.g., attached via cell surface molecules such as integrins to an activated gold surface of the electrode, and micro wells to contain and isolate neurons in the array for subsequent exposure to pain agonist and/or antagonist molecules.

In one embodiment, an electronic neuron pain assay is provided comprising a multielectrode array, co-cultures of neurons and astrocytes bonded to the electrode array, e.g., attached via cell surface molecules such as integrins to an activated gold surface of the electrode, and micro wells to contain and isolate neurons in the array for subsequent exposure to pain agonist and/or antagonist molecules.

In one embodiment, an in vitro electronic measuring model of human pain, in particular dorsal root ganglion neurons or a co-culture of neurons and astroytes, is provided. In one embodiment, an in vitro multi electrode electronic measuring model of human pain, e.g., using DRG neurons or a co-culture of human neurons and astrocytes, in an n by n array format when n=1 to 100, is provided. In one embodiment, an in vitro multi electrode electronic measuring model of human pain is provided, e.g., having a co-culture of human neurons and astrocytes, in an n by n array format when n=1 to 100, where each well containing cells is exposed to pain stimuli agents. In one embodiment, an in vitro multi electrode electronic measuring model of human pain is provided having DRG neurons in an n by n array format when n=1 to 100, where each well of the n by n array is exposed to pain reducing compounds after exposure to pain stimuli compounds. In one embodiment, an in vitro multi electrode electronic measuring model of human pain is provided having co-cultures of neurons and astrocytes in an n by n array format when n=1 to 100, where each well of the n by n array is exposed to pain reducing compounds after exposure to pain stimuli compounds.

### BRIEF DESCRIPTION OF THE DRAWINGS

FIG. 1 shows a top view of a multi electrode array (MEA) according to an embodiment of the present subject matter.

FIG. 2 shows a top view of a MEA electrode in a microwell plate according to an embodiment of the present subject matter.

FIG. 3 shows a typical MEA electronic data read out of electrically firing neurons.

### DETAILED DESCRIPTION

The following discussion is directed towards the various embodiments of the invention. Although one or more of these embodiments may be preferred, the embodiments disclosed should not be interpreted, or otherwise used, as a limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

At the present time, there is no in vitro electronic array assay to model human pain. The present subject matter provides, among other things, an in vitro human model of neuron pain, in particular dorsal root ganglion neurons or a co-culture of neurons and astrocytes, for high throughput screening and testing of compounds to treat short term and chronic pain that also may have a low tendency for addiction or side effects.

A microelectrode array (MEA) is a grid of tightly spaced electrodes in a planar array at the bottom of a cell culture plate. Electrically active cells, such as neurons or cardiomyocytes, can be cultured over the electrodes. Over time, as the cultures become established, they form cohesive networks and present an electrophysiological profile. The resulting electrical activity, spontaneous or induced firing of neurons, or the uniform beat of cardiomyocytes, is captured from each electrode on a microsecond timescale providing both temporally and spatially precise data. In contrast to traditional electrophysiology approaches, such as patch clamp, the electrical activity measured on each MEA electrode is the total extracellular change in ions, reported as changes in voltage. Extracellular field potential provides access to electrophysiological data without disrupting the cellular membrane (non-invasive) or requiring dyes (label-free). Thus, MEA recordings can be taken over time on the same culture (hours-long continual data collection or repeated reads of the same plate over days, hours, or months), an advantage traditional techniques cannot provide.

Additionally, each electrode on the microelectrode array is capable of recording or stimulating the overlying cell culture allowing monitoring and control of cellular network behavior in each well. With these capabilities, complex biological networks can be assayed, e.g., to learn how a cell's circuitry functions together, and in turn advancing applications such as disease modeling, stem cell development, drug discovery and safety/toxicity testing. Typical MEA systems that may be used may be obtained from Axion Biosystems (Atlanta Ga.), ACEA Biosystems Inc. (San Diego, Calif.), and Applied Biophysics Inc. (Troy N.Y.).

In one embodiment, a method for detecting electrical activity of electrically active cells exposed to one or more compounds is provided. In one embodiment, a multi-well plate having electrodes in a planar array affixed to the bottom of the wells and electrically active cells disposed thereon is provided. In one embodiment, the electrode is optionally coated with an agent that enhances binding of cells. The cells are exposed to one or more compounds and electrical activity in the cells in one or more wells of the multi-well plate is determined, e.g., measure or monitored over time. In one embodiment, the cells are human neurons or cardiomyocytes. In one embodiment, the neurons are DRGs. In one embodiment, the cells are a co-culture of human neurons and astrocytes. In one embodiment, the cells are derived from iPSc. In one embodiment, the method further comprises recording the electrical activity. In one embodiment, the method further comprises stimulating the cells with the electrodes before the exposure. In one embodiment, the method further comprises stimulating the cells with the electrodes after the exposure. In one embodiment, the electrode comprises an electrode support and conductive electrodes. In one embodiment, the substrate comprises glass, silicon, standard printed circuit board (PCB), or flexible polymeric film. In one embodiment, the film comprises Kapton, polycarbonate, or polyester (PET). In one embodiment, the electrodes are coated with gold. In one embodiment, the electrodes are coated with one or more agents including fibronectin, laminin, REDV or KREDVY. In one embodiment, the mean firing rate or bursting is quantified. In one embodiment, the one or more compounds increase electrical activity and optionally the cells are subsequently exposed to one or more other compounds. In one embodiment, the one or more compounds decrease electrical activity and optionally the cells are subsequently exposed to one or more other compounds.

Further provided is a multi-well plate having electrodes in a planar array affixed to the bottom of the wells, wherein the electrode is coated with a noble metal or an agent that enhances binding of cells, and wherein electrically active cells comprising human neurons are disposed on the coated electrode. In one embodiment, the metal comprises gold. In one embodiment, the cells are DRG neurons. In one embodiment, the cells comprise neurons and astrocytes.

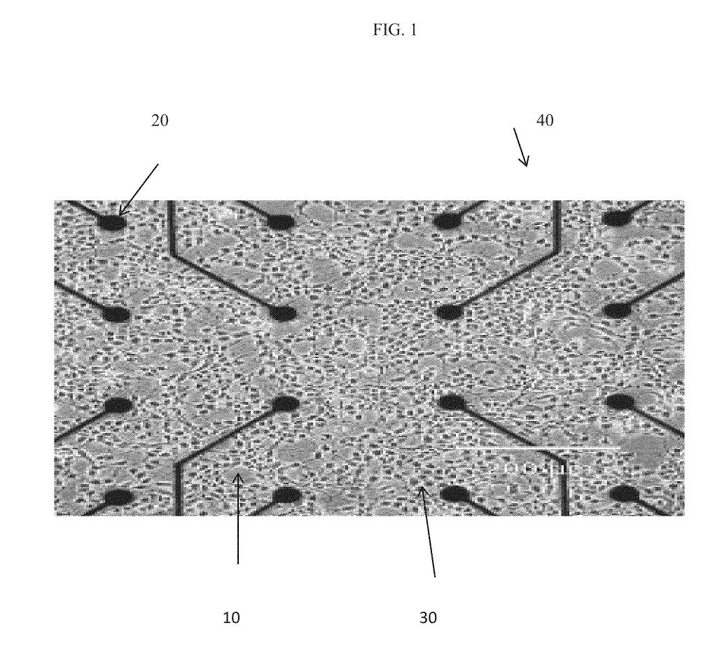

FIG. 1 shows a top view of a multi electrode array (MEA) according to an embodiment of the present subject matter. The MEA 40 may be comprised of an electrode support 10, conductive electrodes 20, and neurons 30 of interest. The electrode support 10 may be formed of glass, silicon, standard printed circuit board (PCB), or flexible polymeric film such as Kapton, polycarbonate, or polyester (PET) film. The thickness of the support 10 may, in one embodiment, range from about 1 micron to about 2 millimeters, e.g., about 25 to about 250 microns. The support 10 may be, in one embodiment, opaque or transparent, e.g., a transparent PET. The conductive electrodes 20 may be comprised of a conductor such as copper, silver, gold, nickel, aluminum, indium tin oxide, graphene, carbon nanotubes, carbon nanobuds, and silver nanowires. The electrodes 20 may have, in one embodiment, an electrical resistivity of less than 100 ohms per square, e.g., less than 10 ohms per square. The electrodes may be patterned in any geometric shape or size, e.g., width lines and interdigitated conductive lines. The width of the lines may vary from about 1 to about 300 microns, e.g., about 50 to about 100 microns. In one embodiment, copper electrodes 10 that have been flash plated with gold to make the surface more biologically compatible for cell attachment and viability are employed. Once the multielectrode array 40 has been fabricated, by techniques well known in the prior art, on a support material 10 neurons are disposed on, e.g., placed in contact with, the gold electrodes.

Good cell adhesion and attachment allow for cell functioning, viability and measurement of the electrophysiology of the neurons during drug exposures of the stack. In one embodiment, gold-coated electrodes 20 may be plasma cleaned to remove any surface contamination and then reacted with a 20 mM solution of alkanethiols of 11-mercaptoundecanoic acid (MUA) for about 5 to about 10 minutes. This results in a self assembled monolayer (SAM) or MUA on the surface. The electrodes may then be immersed into a 150 mM solution of 1-ethyl-3-(3-dimethylamino-propyl) carbodiimide (EDAC) and 30 mM N-hydroxysuccinimide (NHS) for 30 minutes to attach the NHS group to the terminus —COOH of the SAM layer. The finished activated electrode structure may then be sterilized with 70% ethanol for 15 minutes and, in one embodiment, exposed to various proteins that have binding sites for cells. For example, the protein or polypeptides may be fibronectin, laminin, Arg-Glu-Asp-Val-Tyr (REDV) or Lys-Arg-Glu-Asp-Val-Try (KREDVY). In one embodiment KREDVY is employed for cell binding and viability after cell attachment. Neurons 30 are subsequently cultured on the metal plated, e.g. gold-plated, and/or protein-activated electrodes 20. There are many types of human neurons that may be used, such as those derived from primary cells, or those derived from iPScs. There are about 10,000 specific types of neurons in the human brain but generally speaking they can be classified as motor neurons, sensory neurons, and interneurons. In one embodiment, DRGs are employed because they play a role in the detection and transmission of pain. In one embodiment, iPSc derived DRGs are in the neuron 30 layer. In one embodiment, co-cultures of neurons and astrocytes are employed. In one embodiment, co-cultures of neurons and astrocytes are in the neuron 30 layer.

In an embodiment, iPSc derived DRG cells are cultured on top of the MEA gold or noble metal coated electrodes. In an embodiment, DRG cells are fabricated using the protocols used by Dib-Hajj et al., Pain, September 2014, Volume 155, pages 1681-1682. The DRG cells may be bonded to the electrode surface using the aforementioned protocol. Other fabrication and bonding approaches may be used without departing from the scope of the present subject matter.

In an embodiment, co-cultures of neurons and astrocytes are cultured on top of the MEA gold or noble metal coated electrodes. The cells may be attached to the electrode surface using coatings, e.g., of one or more peptides. Other fabrication and bonding approaches may be used without departing from the scope of the present subject matter.

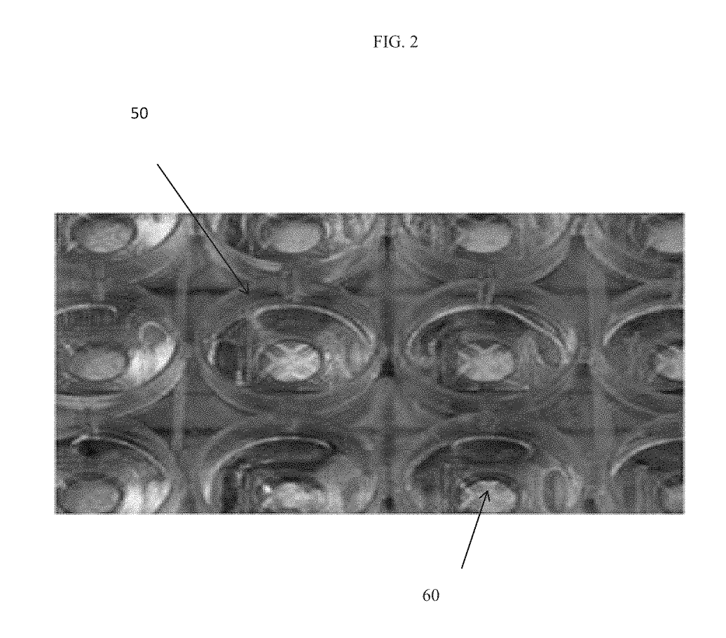

FIG. 2 shows a top view of a MEA electrode in a microwell plate according to an embodiment of the present subject matter. MEA 40 is attached to microwell cell chambers 50. The chambers 50 contain the cell support media for growth and viability. The number of chambers 50 may range from n=1 to an array of n=100 by 100. However, in one embodiment 96 or 384 wells are used. The chambers not only support the cell growth and viability but also isolate the MEAs from each other so that each well can act as an individual test or reaction chamber.

Into each well 60 a compound or compounds may be added and the electrical response of the cells in each well monitored in real time to assess the efficacy or toxicity of the compounds. The compounds can be small molecules, or biologics such as proteins, enzymes, or antibodies. The concentrations of the agonist(s) or antagonist(s) can be varied in different wells, or in the same well at different time points, e.g., after the cells return to baseline, depending on the electrical response of the cells.

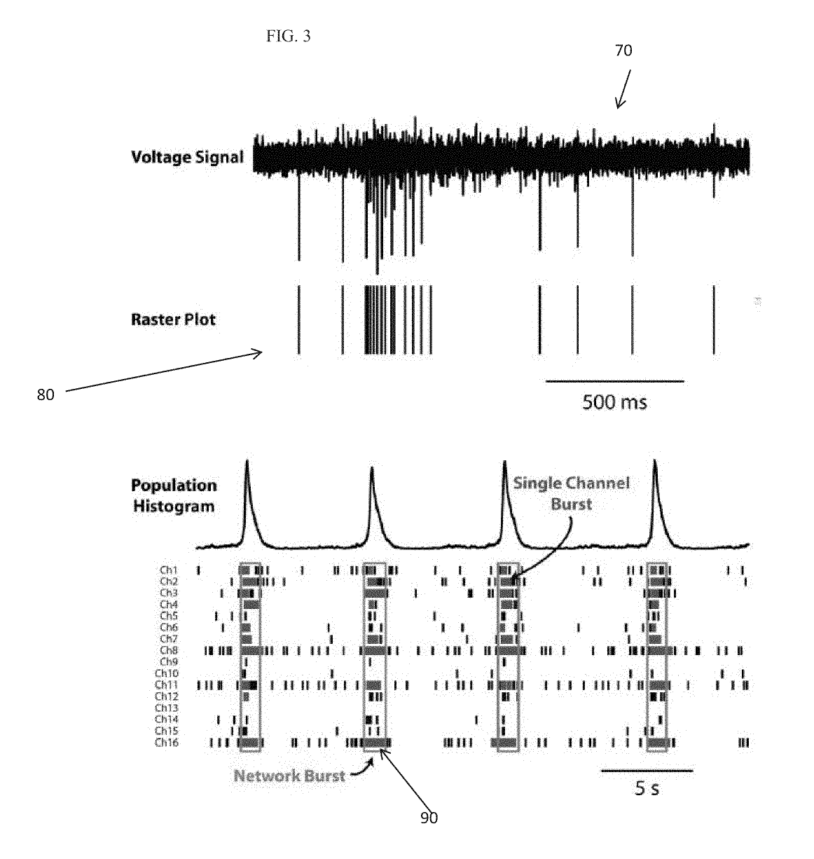

Neurons within the population produce spontaneous action potentials. The mean firing rate (MFR) counts action potentials over time to quantify functionality. Neurons may fire multiple action potentials within a short time period, called a burst (see FIG. 3, 90). Established algorithms detect and quantify burst behavior. Synaptic connections between neurons in a population may lead to coincident action potentials. Network burst and synchrony measurements quantify connectivity.

The timing of the spikes contains all of the information required to calculate measures like mean firing rate (activity over time) or bursting (clusters of action potential activity.

Neural action potentials are detected as changes in voltage above a user-defined threshold as in FIG. 3, 70, Voltage Signal. A simple view of this activity is a raster plot as shown in FIG. 3 Raster Plot 80. Each detected action potential is represented by a “tick” mark to denote the spike time. The plots and graphs in FIG. 3, 70 show typical data recorded for neurons in the basal state before any antagonists or agonists are added to the wells. Upon addition of excitatory molecules that cause pain the electrical activity will increase and have a signature profile. Upon subsequent addition of a molecular agonist or inhibitory agents the electrical behavior will return to basal conditions. Using this protocol the present subject matter can be used to identify pain killing or modulating analgesics.

The above discussion is meant to be illustrative of the principle and various embodiments of the present invention. Numerous variations and modifications will become apparent to those skilled in the art once the above disclosure id fully appreciated. It is intended that the following claims be interpreted to embrace all such variations and modifications.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method for detecting electrical activity of electrically active cells exposed to one or more compounds, comprising:
providing a multi-well plate having electrodes in a planar array affixed to the bottom of the wells, wherein the electrode is optionally coated with an agent that enhances binding of cells, and wherein the electrode has electrically active cells disposed thereon;
exposing the cells to one or more compounds; and
detecting electrical activity in the cells in one or more wells of the multi-well plate.

2. The method of claim 1 wherein the cells are human neurons or cardiomyocytes.

3. The method of claim 2 wherein the neurons are DRGs.

4. The method of claim 1 wherein the cells are a combination of human neurons and human astrocytes.

5. The method of claim 1 wherein the cells are derived from iPSc.

6. The method of claim 1 further comprising recording the electrical activity.

7. The method of claim 1 further comprising stimulating the cells with the electrodes before the exposure.

8. The method of claim 1 further comprising stimulating the cells with the electrodes after the exposure.

9. The method of claim 1 wherein the electrode comprises an electrode support and conductive electrodes.

10. The method of claim 9 wherein the substrate comprises glass, silicon, standard printed circuit board (PCB), or flexible polymeric film.

11. The method of claim 10 wherein the film comprises Kapton, polycarbonate, or polyester (PET).

12. The method of claim 1 wherein the electrodes are coated with gold or with one or more agents including fibronectin, laminin, REDV or KREDVY.

13. The method of claim 1 wherein mean firing rate or bursting is quantified.

14. The method of claim 1 wherein the one or more compounds increase electrical activity.

15. The method of claim 14 wherein the cells are subsequently exposed to one or more other compounds.

16. The method claim 1 wherein the one or more compounds decrease electrical activity.

17. The method of claim 16 wherein the cells are subsequently exposed to one or more other compounds.

18. A multi-well plate having electrodes in a planar array affixed to the bottom of the wells, wherein the electrode is coated with a noble metal or an agent that enhances binding of cells, and wherein electrically active cells comprising human neurons are disposed on the coated electrode.

19. The plate of claim 18 wherein the metal comprises gold.

20. The plate of claim 18 wherein the cells are DRG neurons or a combination of neurons and astrocytes.
